(19) United States
(12) Patent Application Publication
(10) Pub. No.: US 2026/0245179 A1
(43) Pub. Date: Aug. 20, 2026

(54) IMAGING DEVICE AND CONTROL METHOD OF IMAGING DEVICE

(71) Applicant: CANON KABUSHIKI KAISHA, Tokyo (JP)

(72) Inventor: JUNYA FUKUMOTO, Kanagawa (JP)

(21) Appl. No.: 19/544,327

(22) Filed: Feb. 19, 2026

(30) Foreign Application Priority Data
Feb. 19, 2025 (JP) 2025-024787

(51) Int. Cl. G06T 5/50; G01N 21/64; H04N 23/13; H04N 23/72; H04N 23/73; H04N 23/74; H04N 25/531

(52) U.S. Cl. CPC G06T 5/50; G01N 21/6456; H04N 23/13; H04N 23/72; H04N 23/73; H04N 23/74; G06T 2207/10024; G06T 2207/10064; G06T 2207/10152; G06T 2207/20221; G06T 2207/20224; G06T 2207/30004; H04N 25/531

(57) ABSTRACT

An imaging device is provided that includes a light source module configured to output visible light and excitation light, an imaging module including at least one image sensor, a processor, and a memory storing a program. When executed by the processor, the program causes the imaging device to acquire a visible image of a subject using the visible light, a fluorescent image of fluorescence generated in the subject using the excitation light, and an ambient image of the subject using ambient light, and to generate and output composite images at a prescribed output cycle. The program controls acquisition timing so that fluorescent image acquisition and ambient image acquisition are alternately executed between successive visible image acquisitions, and generates composite images using visible, fluorescent, and ambient images acquired before and after respective visible image acquisitions.

### BACKGROUND

### Field of the Technology

The present disclosure relates to an imaging device that enables fluorescence observation under ambient light.

### Description of the Related References

Fluorescence observation is widely used particularly in the medical field. For example, in laparoscopic surgery, indocyanine green (ICG), which is a fluorescent substance, is introduced into the blood stream, and fluorescence obtained by irradiating excitation light of a specific wavelength is observed. This increases the visibility of blood flow that is difficult to be seen under ordinary visible light observation, thereby enabling the prevention of suture failure and improving the accuracy of identifying an excision range.

In abdominal surgery as well, there is a need to use ICG for the same purpose. However, because the fluorescence wavelength of ICG lies in the near-infrared region and cannot be seen with the naked eye, it is essential to use an imaging device sensitive to near-infrared light. In addition, when an image obtained under visible light observation and an image obtained under fluorescence observation are combined and displayed, the position of a fluorescent portion can not always be easily identified. Therefore, it is more desirable to use an imaging device sensitive to near-infrared light and also to visible light. Such combination generally employs a process in which a pseudo-colored fluorescent marker is superimposed on the fluorescent portion on the basis of a visible light image.

However, when an imaging device sensitive to both visible light and near-infrared light is used in abdominal surgery, the fluorescent portion cannot be correctly recognized because the imaging device is affected by ambient light, such as lighting in the operating room or shadowless lamps illuminating the surgical site. Therefore, a technology has been proposed in which images are sequentially acquired in a time-sharing manner in a cycle of a visible light image, an ambient light image, and a fluorescent image, and the fluorescent image is compensated using the ambient light image, thereby suppressing the influence of the ambient light (e.g., Japanese Patent Application Laid-open No. 2021-191418).

However, when the visible light image, the ambient light image, and the fluorescent image are sequentially acquired in a time-sharing manner and a combined image is output, the frame rate of the visible light image may fall below a desired rate.

### SUMMARY

The present disclosure prevents or suppresses a decrease in the frame rate of visible light images while enabling fluorescence observation under ambient light, thereby providing moving images suitable for users.

The present disclosure provides an imaging device comprising: a light source module capable of outputting visible light and excitation light; an imaging module having at least one image sensor; a processor; and a memory storing a program which, when executed by the processor, causes the imaging device to control the light source module and the imaging module, thereby enabling visible image acquisition processing to capture a visible image representing an image of a subject using visible light output from the light source module, fluorescent image acquisition processing to capture a fluorescent image representing an image of fluorescence generated in the subject using excitation light output from the light source module, and ambient image acquisition processing to capture an ambient image representing an image of the subject using ambient light, and to execute image processing to generate a composite image using the visible image, the fluorescent image, and the ambient image and to output the composite image at a prescribed output image cycle, wherein the visible image acquisition processing is executed at a cycle equal to or shorter than the output image cycle, wherein executing at least the fluorescent image acquisition processing during a period between the visible image acquisition processing and the next visible image acquisition processing and executing at least the ambient image acquisition processing during a period between the visible image acquisition processing and the next visible image acquisition processing are alternately repeated, and wherein the image processing executes processing to generate a first composite image using at least a first visible image acquired in first visible image acquisition processing, a fluorescent image acquired during a period after the first visible image acquisition processing, and an ambient image acquired during a period before the first visible image acquisition processing, and processing to generate a second composite image using at least a second visible image acquired in second visible image acquisition processing, a fluorescent image acquired during a period before the second visible image acquisition processing, and an ambient image acquired during a period after the second visible image acquisition processing.

Features of the present disclosure will become apparent from the following description of embodiments with reference to the attached drawings. The following description of embodiments is described by way of example.

### BRIEF DESCRIPTION OF THE DRAWINGS

FIG. 1 illustrates a configuration example of an imaging device according to a first embodiment.

FIG. 2 illustrates an example of the spectral characteristics of illumination and fluorescence.

FIG. 3 illustrates an example of the spectral characteristics of an optical filter.

FIG. 4 illustrates an example of the spectral characteristics of an imaging element that incorporates a color filter.

FIG. 5 illustrates an example of the timing of illumination, exposure, and image output according to the first embodiment.

FIG. 6 illustrates an example of the timing of illumination, exposure, and image output in a comparative example.

FIG. 7 illustrates a configuration example of an imaging device according to a second embodiment.

FIG. 8 illustrates an example of the spectral characteristics of a color separation element.

FIG. 9 illustrates an example of the spectral characteristics of imaging elements.

FIG. 10 illustrates an example of the timing of illumination, exposure, and image output according to the second embodiment.

FIG. 11 illustrates an example of the timing of illumination, exposure, and image output according to a third embodiment.

FIG. 12 illustrates an example of the timing of illumination, exposure, and image output according to a fourth embodiment.

FIG. 13 illustrates a configuration example of an imaging device according to a fifth embodiment.

FIG. 14 illustrates an example of the spectral characteristics of the color separation element.

FIG. 15 illustrates an example of the timing of illumination, exposure, and image output according to the fifth embodiment.

FIG. 16 illustrates an example of the spectral characteristics of the color separation element according to a modified example of the fifth embodiment.

### DESCRIPTION OF THE EMBODIMENTS

When a visible light image, an ambient light image, and a fluorescent image are sequentially acquired in a time-sharing manner and a combined image is output, the frame rate of the visible light image may fall below a desired rate. For example, when the maximum frame rate of the imaging element (image sensor) that is a component of an imaging device is 120 fps, the frame rate of the visible light image becomes 40 fps. However, in medical applications, a frame rate 50 fps or higher is often required. Because the main component of the composite image is the visible light image, a low frame rate of the visible light image may cause a sense of discomfort for a user.

Furthermore, because a certain time lag always occurs between the visible light image and the fluorescent image, the positions of the visible light image and the fluorescent marker deviate from those of moving subjects. For example, when the fluorescent portion reciprocates at an appropriate speed, a moving image may be generated in which the motion of the fluorescent marker lags behind the visible light image, causing a sense of discomfort for the user.

Hereinafter, embodiments of the present disclosure will be described in detail by way of example with reference to the drawings.

### First Embodiment

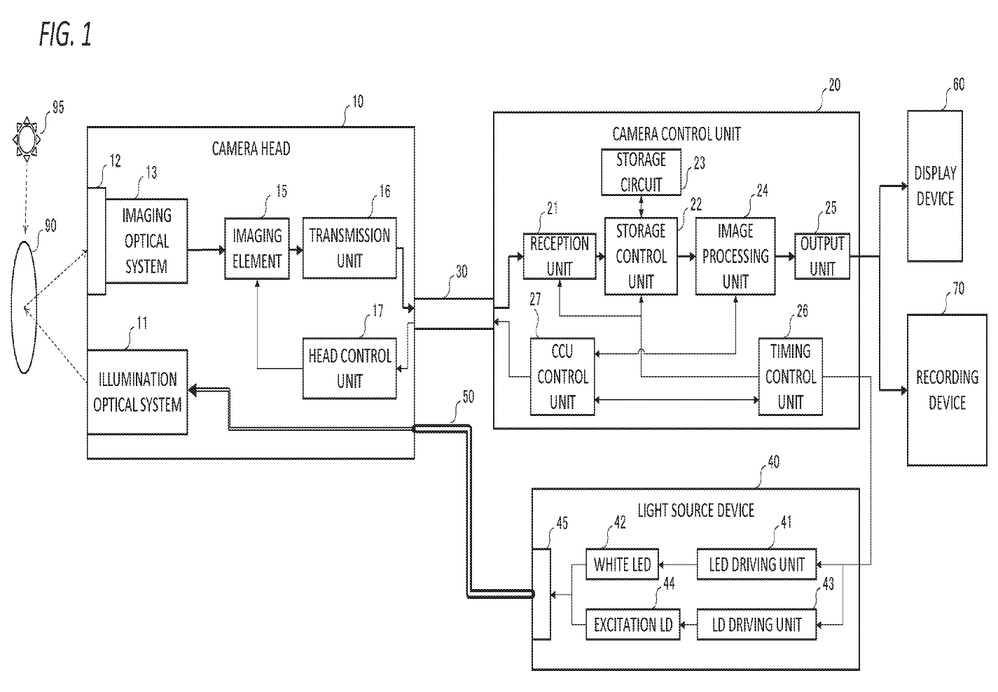

FIG. 1 is a diagram illustrating a configuration example of an imaging device according to a first embodiment.

The imaging device of this embodiment has, as its main components, a camera head 10, a camera control unit (CCU) 20, and a light source device 40. Light emitted from the light source device 40 is transmitted to the camera head 10 via a light guide cable 50 and irradiated toward a subject 90 through an illumination optical system 11. The resulting reflected light and fluorescence pass through an excitation-light cut filter 12 and an imaging optical system 13 incorporated in the camera head 10 and are formed into an image on an imaging element 15.

At this time, in an environment such as abdominal surgery, unintended reflected light from an ambient light source 95 is also similarly formed into an image on the imaging element 15 substantially constantly.

An image signal output from the imaging element 15 is output from the camera head 10 via a transmission unit 16 and is transmitted to the CCU 20 via a camera cable 30. In the CCU 20, the input image signal is received by a reception unit 21 and input to a storage control unit 22. The storage control unit 22 adjusts the timing of the image signal using a storage circuit 23 and inputs the necessary image signal to an image processing unit 24. The image signal processed by the image processing unit 24 is output from the CCU 20 via an output unit 25 and is input to a display device 60 and a recording device 70 to be displayed and recorded.

The CCU 20 has a timing control unit 26. The timing control unit 26 collectively and synchronously controls the irradiation timing of the light source device 40, the driving timing of the camera head 10, the storage and readout timing of the storage control unit 22, and the like.

Instructions from the outside are received by a CCU control unit 27. The CCU control unit 27 appropriately changes the settings or operating modes of the timing control unit 26, the image processing unit 24, the camera head 10, and the like in accordance with the instructions. When changing the control of the camera head 10, the CCU control unit 27 provides instructions to a head control unit 17. Upon receiving the instructions from the CCU control unit 27, the head control unit 17 changes the settings or driving timing of the imaging element 15 as appropriate.

The light source device 40 has a white light-emitting diode (LED) 42 that outputs visible light and an excitation laser diode (LD) 44 that outputs excitation light, and is capable of outputting both visible light and excitation light. The light source device 40 combines the emitted light from the white LED 42 and the excitation LD 44 using an optical adapter 45 and outputs the combined light to the guide cable 50. The timing control unit 26 of the CCU 20 outputs a control signal to the light source device 40 as appropriate. In response to the control signal, an LED driving unit 41 and an LD driving unit 43 adjust the lighting timing or output level of the white LED 42 and the excitation LD 44, respectively.

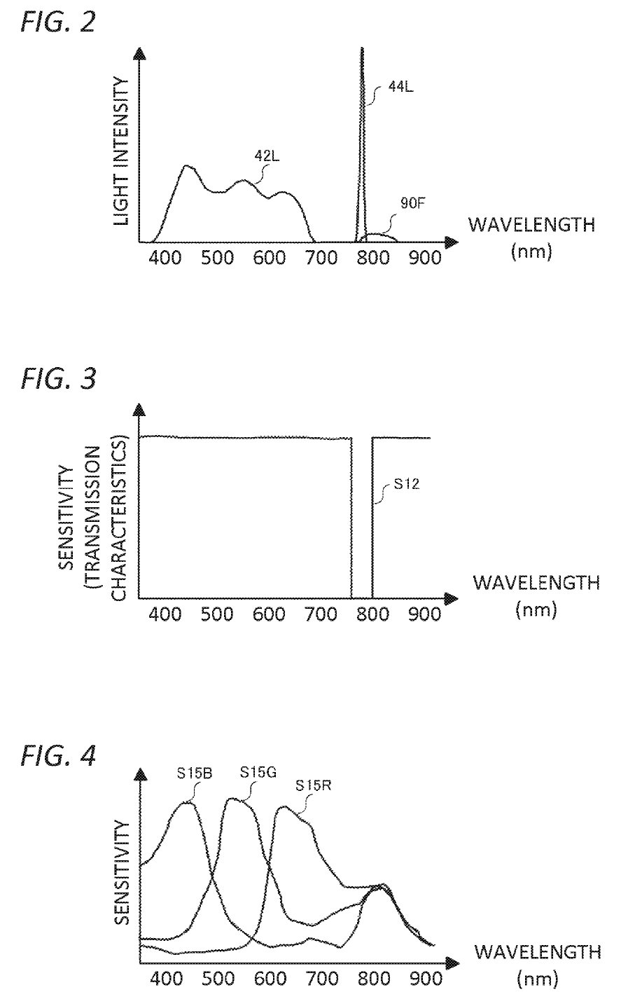

FIG. 2 illustrates an example of the spectral characteristics of illumination and fluorescence. The white LED 42 irradiates visible light 42L, which is widely distributed over 400 nm to 700 nm. The excitation LD 44 irradiates intense narrow-band light 44L near 780 nm, which corresponds to the excitation peak of ICG. The subject 90 is previously administered ICG via blood so that a portion of the subject 90 contains ICG. When the subject 90 is irradiated with the light 44L from the excitation LD 44, fluorescence 90F having a wavelength slightly longer than that of the excitation light 44L is generated. In general, the intensity of the fluorescence 90F is weaker than that of the excitation light 44L or the white light 42L. Although not explicitly illustrated, the ambient light source 95 is considered to have wavelength characteristics similar to those of the white LED 42, for example.

FIG. 3 illustrate an example of the spectral characteristics of the excitation-light cut filter 12. The excitation-light cut filter 12 has characteristics S12 that block the wavelength near 780 nm to cut the wavelength of the excitation light 44L output from the excitation LD 44, and that transmit the other wavelengths. By passing through this excitation-light cut filter 12, the reflected portion of the excitation light 44L is blocked, preventing the excitation light 44L from mixing with the fluorescence 90F.

FIG. 4 illustrates an example of the spectral characteristics of the imaging element 15. In this example, the imaging element incorporates a color filter and exhibits one of the characteristics S15B, S15G, and S15R depending on the pixel. The color filter is configured, for example, in a Bayer arrangement in which blue, green, and red pixels are arranged at fixed intervals. Therefore, a color image can be formed, for example, by interpolation using the image processing unit 24.

The pixels of all the characteristics are sensitive to the wavelength of the fluorescence 90F and also have a certain degree of sensitivity to the white light 42L of the white LED 42 and the ambient light 95L from the ambient light source 95. Therefore, the fluorescence 90F alone cannot be obtained when the white LED 42 or the ambient light source 95 is lit. In addition, the ambient light source 95 is outside of the control range of the imaging device and is generally constantly irradiated. Therefore, it is not possible to acquire the fluorescence 90F alone. The intensity of the fluorescence 90F is generally weak, and the influence of the ambient light 95L mixed in cannot be ignored. Therefore, when it is desired to obtain the intensity of the fluorescence 90F, it is necessary to reduce the influence of the ambient light 95L by some means.

Similarly, it is not possible to acquire the reflected light of the white LED 42 alone. However, in many cases, the intensity of the light 42L from the white LED 42 is sufficiently higher than that of the ambient light 95L. Therefore, for a visible image, the influence of the ambient light 95L mixed in may often be ignored. Even if the ambient light 95L has non-ignorable intensity, it can be useful for the influence of the ambient light 95L to not be removed because the total of the reflected light from the white LED 42 and the reflected light from the ambient light 95L corresponds to the image actually seen.

FIG. 5 illustrates an example of the timing of illumination, exposure, and image output according to this embodiment.

The processing described below is executed when the control unit (the CCU control unit 27, the storage control unit 22, and the timing control unit 26) of the CCU 20 controls the light source device 40 and the imaging unit (imaging module) of the camera head 10. Here, an image of the subject 90, which is captured by the imaging unit using the white light (visible light) 42L output from the light source device 40, is referred to as a “visible image,” and processing for acquiring a visible image is referred to as “visible image acquisition processing.” An image of the fluorescence 90F, which is generated in the subject 90 by the excitation light 44L output from the light source device 40 and captured by the imaging unit, is referred to as a “fluorescent image,” and processing for acquiring a fluorescent image is referred to as a “fluorescent image acquisition processing.” Furthermore, an image of the subject 90, which is captured by the imaging unit using the ambient light 95L, is referred to as an “ambient image,” and processing for acquiring an ambient image is referred to as an “ambient image acquisition processing.” The same applied to the following embodiments.

It is assumed that times T1, T2, T3, etc., are arranged at 1/120-second intervals. In accordance with instructions from the timing control unit 26, the white LED 42 repeats, every 1/120 second, a 2/120-second cycle in which it is lit at time T1, extinguished at time T2, lit at time T3, and extinguished at time T4 in an alternating manner. The excitation LD 44 repeats a cycle in which it is lit at time T2, extinguished from time T3 to time T5, lit at time T6, and is extinguished from time T7 to time T9 in a 4/120-second cycle. For 1/120 second within each 4/120-second cycle, such as time T4 or time T8, there occurs a timing at which both the white LED 42 and the excitation LD 44 are extinguished.

Here, a case is considered in which the imaging element 15 is a global-shutter-type image sensor sensitive to a wavelength range from visible light to the near-infrared range of fluorescence (near-infrared light). Typical examples of sensors include CCD image sensors and some CMOS image sensors. The sensor that is exposed to the light reflected from the white LED 42 at time T1 outputs the resulting visible image W1 at the next time T2 (visible image acquisition processing). The exposure and the visible image W1 at time T1 contain the reflected light of the ambient light 95L. Similarly, the sensor that receives the fluorescence 90F generated by the excitation LD 44 during the exposure period at time T2 outputs the resulting fluorescent image F2 at the next time T3 (fluorescent image acquisition processing). The fluorescent image F2 also contains the reflected light of the ambient light 95L. Because exposure occurs at the timing when both light sources are extinguished at time T4, only the reflected light of the ambient light 95L is contained in the ambient image A4 output at time T5 (ambient image acquisition processing). As a result, the sensor output repeats a cycle of a visible image Wn, a fluorescent image Fn+1, a visible image Wn+2, and ambient image An+3.

Here, the letters W, F, and A represent “visible image,” “fluorescent image,” and “ambient image,” respectively, and the subscripts n, n+1, n+2, n+3, etc., represent exposure times (frame numbers). For example, Wn+2 represents a visible image exposed during the period of the (n+2)-th frame. The same applies to the following figures.

The storage control unit 22 stores these periodic image signal outputs from the sensor in the storage circuit 23 and separately outputs them to the image processing unit 24 once necessary information is obtained. The image processing unit 24 generates a composite image using the received visible image, fluorescent image, and ambient image, and outputs the generated composite image at a prescribed output image cycle. Here, the desired composite result is an image in which the portion containing ICG is marked with a pseudo color relative to the visible image. In many cases, the pseudo color is selected from highly saturated colors easily distinguishable from the visible image, blue or green hues that generally do not exist in the human body that is an operated target, or colors combining these characteristics. Here, it is desirable that the image processing unit 24 detect the portion containing ICG on the basis of the intensity of the fluorescence 90F. However, the fluorescent image contains not only the component of the fluorescence 90F but also the reflection component of the ambient light 95L. Therefore, the image processing unit 24 estimates only the component of the fluorescence 90F by also referring to the ambient image. For example, in processing for generating a composite image, the image processing unit 24 may reduce an ambient light component contained in a fluorescent image by subtracting an ambient image from the fluorescent image. In this case, it is not necessary to completely remove the ambient light component contained in the fluorescent image. It is sufficient to reduce the ambient light component to a negligible degree relative to the fluorescent component. That is, the fluorescent component contained in the fluorescent image needs to be emphasized. By superimposing the fluorescent image in which the ambient light component has been reduced on a visible image using a pseudo color, a composite image in which the portion containing ICG is emphasized can be obtained.

Here, because there is a time lag between the fluorescent image and the ambient image, it is desirable to refer to an image captured at a time as close as possible to perform subtraction. Therefore, in this example, the ambient image An-2 or An+2 is subtracted from the fluorescent image Fn to calculate fluorescent intensity. In addition, because it is desirable that the time lag from the visible image be small, an output image is generated by combining three images, i.e., the ambient image An-2, the visible image Wn-1, and the fluorescent image Fn, or by combining three images, i.e., the fluorescent image Fn, the visible image Wn+1, and the ambient image An+2. Furthermore, relative to the sensor output of 120 fps, the output image cycle of the composite image is set to 60 fps, which is half of the sensor output.

A specific example will be described below. At time T3, the ambient image A0 and the visible image W1 have already been stored in the storage circuit 23. In addition, the fluorescent image F2 has also been received from the sensor, and its storage has begun. Therefore, the storage control unit 22 simultaneously reads out the ambient image A0, the visible image W1, and the fluorescent image F2 from time T3 to time T4 at 60 fps and inputs the read-out images to the image processing unit 24. The image processing unit 24 generates a fluorescent intensity image in which the component of the fluorescence 90F, which is obtained by subtracting the ambient image A0 from the fluorescent image F2, is represented in a pseudo color. Then, the image processing unit 24 superimposes the fluorescent intensity image on the visible image W1 to generate the desired output image and outputs the resulting image from time T3 to time T4 at 60 fps. Furthermore, from time T5 to time T6, the storage control unit 22 reads out the three images F2, W3, and A4 from the storage circuit 23 at 60 fps, and the image processing unit 24 superimposes a fluorescent intensity image, which is obtained by subtracting the ambient image A4 from the fluorescent image F2, on the visible image W3, and outputs the resulting image at 60 fps.

Note that even when images captured at times as close as possible are combined, the time lag does not become zero. Therefore, motion estimation may be performed to correct the time lag. For example, by searching for corresponding pixels between two images at different times (frames), a motion vector is obtained for each pixel or block. Then, after correcting the time lag between the two images by shifting the positions of the pixels or blocks of one image so as to cancel the motion vectors, the two images are combined. As a result, a high-quality composite image can be obtained. Furthermore, for the purpose of improving the estimation accuracy or reducing noise, it is also possible to perform image processing on various images while referring to images older than those in the above example.

When subtracting an ambient image from a fluorescent image, it is basically desirable that the imaging conditions of the imaging element, such as gain and exposure time, be set to the same values. However, the same conditions may not always be maintained, for example, during automatic brightness control. In this case, subtraction is performed after generating images as if they had been captured under the same conditions by applying a correction gain to one of the images.

Although the irradiation timing of the white light or excitation light is illustrated as the maximum range for simplicity, the lighting timing may be shortened within that range when the amount of the light is sufficient. Alternatively, the exposure time may be limited by using the shutter function of the sensor, or automatic brightness control may be performed using these functions.

The features of control of the imaging device according to this embodiment as described above can be summarized as follows. The control unit (22, 26, and 27) can execute the visible image acquisition processing to capture a visible image, the fluorescent image acquisition processing to capture a fluorescent image, and the ambient image acquisition processing to capture an ambient image by controlling the light source device (40) and the imaging unit (15). The image processing unit (24) generates a composite image using the visible image, the fluorescent image, and the ambient image, and outputs the composite image at a prescribed output image cycle (60 fps). The control unit (22, 26, and 27) executes the visible image acquisition processing at a cycle equal to or shorter than the output image cycle. Furthermore, the control unit (22, 26, and 27) performs control to alternately repeat: executing at least the fluorescent image acquisition processing (F2) during the period between the visible image acquisition processing (W1) and the next visible image acquisition processing (W3); and executing at least the ambient image acquisition processing (A4) during the period between the visible image acquisition processing (W3) and the next visible image acquisition processing (W5). The image processing unit (24) then generates a first composite image (W1 + (F2 - A0)) using at least a first visible image (W1) acquired in the first visible image acquisition processing, a fluorescent image (F2) acquired during the period after the first visible image acquisition processing, and an ambient image (A0) acquired during the period before the first visible image acquisition processing. Furthermore, the image processing unit (24) generates a second composite image (W3 + (F2 - A4)) using at least a second visible image (W3) acquired in the second visible image acquisition processing, the fluorescent image (F2) acquired during the period before the second visible image acquisition processing, and an ambient image (A4) acquired during the period after the second visible image acquisition processing. The first composite image and the second composite image are alternately output at 60 fps.

With these features, two major advantages can be obtained. The first advantage is that the image output and its main component, the visible image, can be output at 60 fps. As described above, a frame rate of 50 fps or higher is often required in medical applications, and the output of the imaging element is limited to 120 fps or lower in many cases. That is, an output at 60 fps can achieve both of these demands. A fluorescent marker corresponds to a frame rate of 30 fps, but this is comparatively less significant because surgical instruments such as forceps, which require fine movements, do not contain fluorescence.

The second advantage is that the time lag between the base visible image and a fluorescent image or an ambient image can be canceled over the long term. Specifically, for example, because the fluorescent image F2 is combined with the visible image W1, the fluorescent image is temporally ahead of the visible image by 1/120 second. However, in the next output image, because the fluorescent image F2 is combined with the visible image W3, the fluorescent image is delayed from the visible image by 1/120 second. Subsequently, because the fluorescent image F6 is combined with the visible image W5, the fluorescent image is again ahead. Accordingly, in an image containing a moving fluorescent object, in a strict sense, the fluorescent marker is alternately shifted forward and backward by 1/120 second relative to its original position for each frame. However, when such an image is actually viewed as a 60-fps moving image, the marker merely appears slightly blurred in the motion direction as if it has not shifted from its original position due to visual complementation and correction. That is, there is an advantage that the time lag between the visible image and the fluorescent image is visually canceled. When combining two images, it is desirable to completely correct the time lag by motion estimation but it is actually difficult to completely correct it. Accordingly, as in this embodiment, if the combination of images to be combined is set so that the direction of the time lag periodically changes, a sense of discomfort can be reduced even when an error occurs in motion estimation. For example, when the exposure time is limited by a shutter, the temporal center of gravity is not always perfectly aligned, but the advantage of reducing discomfort remains unchanged.

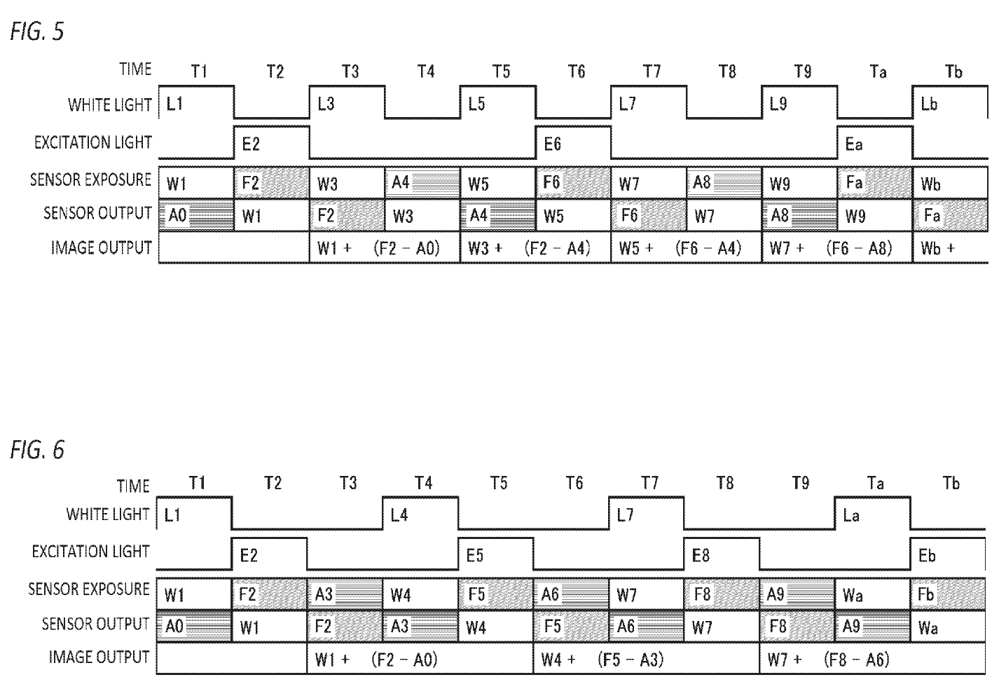

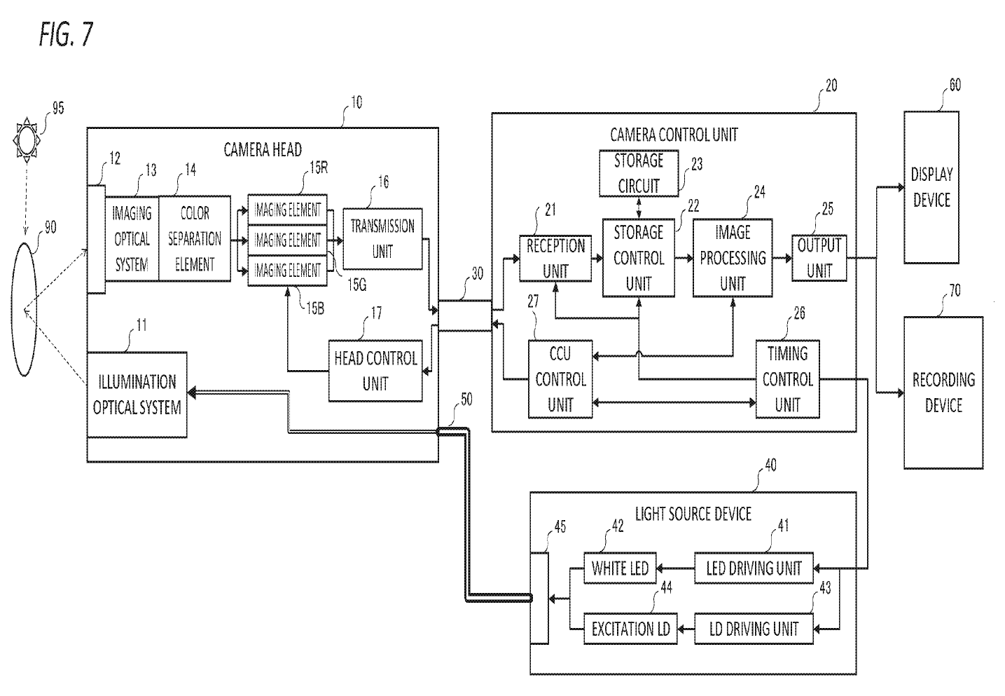

FIG. 6 illustrates an example of the timing of illumination, exposure, and image output in the related art.

The sensor output repeats a cycle of a visible image, a fluorescent image, and an ambient image. Naturally, each image has a frame rate of 40 fps, and the image output has a frame rate of 40 fps. If a frame rate of 60 fps is forcibly realized, it is necessary either to output the same image twice, as in W1 → W4 → W4 → W7, or to generate an interpolated image using advanced motion estimation processing. The former causes unnatural movement because the same image is displayed twice, while the latter increases processing load or device cost. Therefore, general imaging devices cannot support a required frame rate of 50 fps or higher.

As an example, the ambient image A0, the visible image W1, and the fluorescent image F2 are combined, and then the ambient image A3, the visible image W4, and the fluorescent image F5 are combined. In this manner, the time lag between the visible image, the fluorescent image, and the ambient image occurs similarly in all composite images. Therefore, for example, when a fluorescent substance continues to move to the right, the marker continues to shift to the right relative to the visible image, which tends to produce a visually uncomfortable image.

### Second Embodiment

Compared to this comparative example, it is evident that the proposed embodiment provides the above-described two advantages (that a visible image can be output at a frame rate of 50 fps or higher, and that the time lag between images can be apparently canceled).

FIG. 7 illustrates a configuration example of an imaging device according to a second embodiment of the present disclosure. The differences from the first embodiment are that a color separation element 14 is provided inside of the camera head 10, and that three imaging elements (15R, 15G, and 15B) are provided in connection with the color separation element 14. These three imaging elements 15R, 15G, and 15B are of a type that does not incorporate a color filter. This type is generally referred to as a three-chip camera. Furthermore, in this embodiment, it is assumed that these imaging elements 15R, 15G and 15B are rolling-shutter-type image sensors. In the following description, the imaging elements 15R, 15G, and 15B may also be referred to as an “R-sensor,” a “G-sensor,” and a “B-sensor,” respectively.

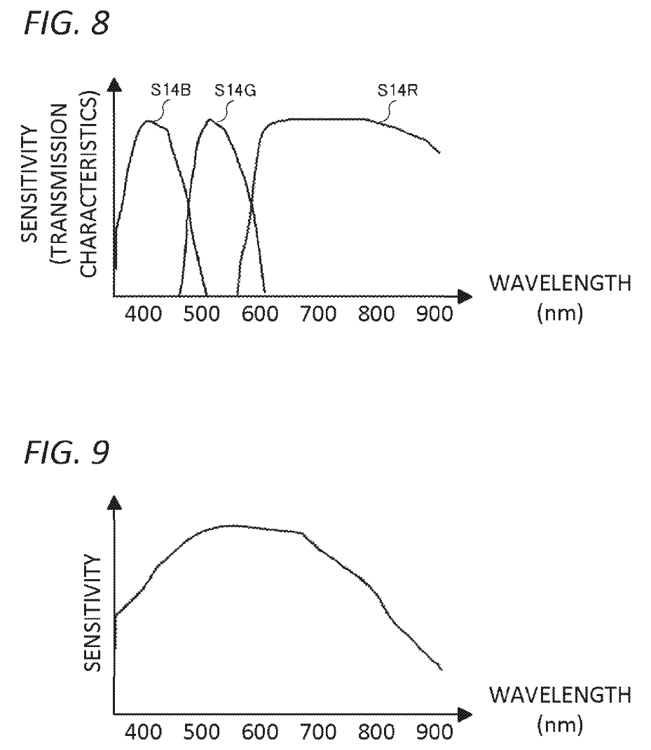

FIG. 8 illustrates an example of the spectral characteristics of the color separation element 14. The color separation element 14 has one incident surface and three emission surfaces. The three emission surfaces are designed to have transmission characteristics indicated by S14B, S14G, and S14R, respectively. Light incident on the incident surface from the imaging optical system 13 is separated into three wavelength components S14B, S14G, and S14R, that is, blue, green, and red light. The light of the wavelength components S14B, S14G, and S14R is incident on the imaging elements 15B, 15G, and 15R, respectively.

FIG. 9 illustrates an example of the spectral characteristics of the imaging elements 15B, 15G, and 15R. In this example, for simplicity, a case is assumed in which the same three CMOS image sensors are used. That is, the three imaging elements 15B, 15G, and 15R each have sensitivity over a wide band from visible light to near-infrared light. However, the present proposal is not limited to this form. For example, different sensors may be combined with each other; an InGaAs sensor may be used only for the imaging element 15R, with importance placed on infrared sensitivity.

The spectral characteristics of the entire imaging device are obtained by combining the spectral characteristics of the color separation element 14, the spectral characteristics of the imaging elements 15B, 15G, and 15R, and the spectral characteristics of the excitation-light cut filter 12. Accordingly, the fluorescence 90F in the near-infrared region passes through the emission surface of the wavelength component S14R of the color separation element 14 and is received by the imaging element 15R. At this time, some wavelength components of the white light 42L and the ambient light 95L are also received by the imaging element 15R. As described above, as in the first embodiment, the imaging element 15R may not acquire the fluorescence 90F alone.

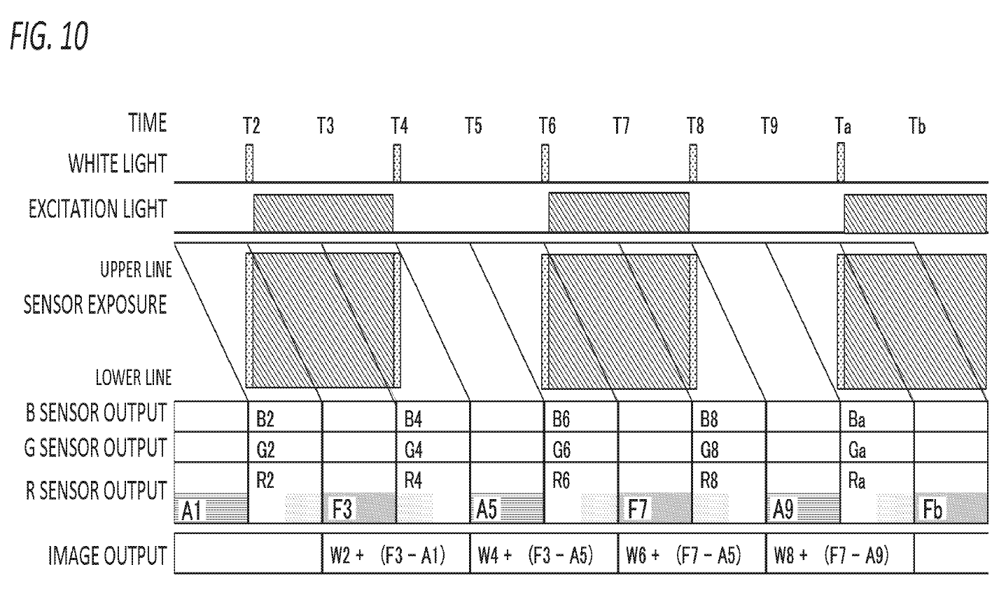

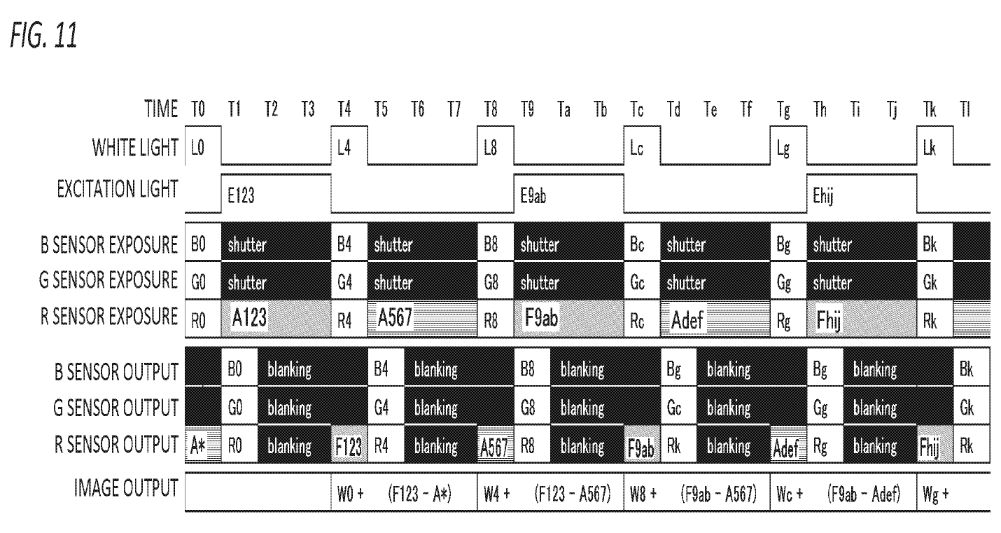

FIG. 10 illustrates an example of the timing of illumination, exposure, and image output according to this embodiment. Because rolling-shutter-type image sensors are used, the difference from the first embodiment is that the exposure timing of the sensors differs for each line.

In FIG. 10, the letter B represents an image (referred to as a “B-image”) output from the B-sensor when the white LED 42 is lit. The letter G represents a “G-image” output from the G-sensor when the white LED 42 lit, and the letter R represents an “R-image” output from the R-sensor when the white LED 42 is lit. Furthermore, the subscripts n, n+1, n+2, n+3, etc., represent exposure times (frame numbers). For example, Bn, Gn, and Rn represent a B-image, a G-image, and an R-image, respectively, that were exposed in the n-th frame period. The same applies to the following figures.

Because the fluorescence 90F is weak, mixing of the white light 42L reflected from the white LED 42 has a significant impact. Therefore, the white LED 42 may be lit only during the blanking period of the sensor or only during a partial period around the blanking period of the sensor (i.e., a period much shorter than one frame period, e.g., equal to or less than 1/10 of one frame period). This lighting control can suppress the mixing of the reflected white light 42L into the fluorescent image as much as possible. For example, for the visible image R2, the white LED 42 is lit only for a very short period around time T2, which corresponds to the blanking period immediately before the exposure period of the frame 2.

In contrast, the fluorescence 90F does not have a significant impact on the visible image, and its fluorescence intensity is desirably increased as much as possible. Therefore, the excitation LD 44 can be controlled so that it remains lit throughout the entire exposure periods of the frames (e.g., F3, F7, and the like) of the fluorescent image. For example, for the fluorescent image F3, the excitation LD 44 is kept lit during the period from time T2 to time T4. At this time, theoretically, the fluorescence 90F is mixed into the visible images R2 and R4 before and after the fluorescent image F3 in a line-by-line gradation manner. However, as described above, because the intensity of the fluorescence 90F is actually extremely weak compared to that of the visible light, the influence of its mixture can be ignored. Even when the intensity is not negligible, the mixture is often overwritten by the fluorescent marker and thus cannot be seen.

Although the control timing of the light source device 40 differs from that of the first embodiment as described above, the obtained sensor outputs remain substantially unchanged, and a cycle of the visible image Wn, the fluorescent image Fn+1, the visible image Wn+2, and the ambient image An+3 is repeated. Here, the visible image Wn represents a color image obtained by combining the B-image Bn, the G-image Gn, and the R-image Rn output from the imaging elements 15B, 15G, and 15R, respectively, when the white LED 42 is lit. Furthermore, the fluorescent image Fn represents an image output from the R-sensor when the white LED 42 is extinguished and the excitation LD 44 is lit, and the ambient image An represents an image output from the R-sensor when both the white LED 42 and the excitation LD 44 are extinguished.

The subsequent image processing flow and the resulting advantages are the same as those in the first embodiment, and their description will be omitted.

The features of control of the imaging device according to this embodiment as described above can be summarized as follows. The control unit (22, 26, and 27) can execute the visible image acquisition processing to capture a visible image, the fluorescent image acquisition processing to capture a fluorescent image, and the ambient image acquisition processing to capture an ambient image by controlling the light source device (40) and the imaging unit (15R, 15G, and 15B). The image processing unit (24) generates a composite image using the visible image, the fluorescent image, and the ambient image, and outputs the composite image at a prescribed output image cycle (60 fps). The control unit (22, 26, and 27) executes the visible image acquisition processing at a cycle equal to or shorter than the output image cycle. Furthermore, the control unit (22, 26, and 27) performs control to alternately repeat: executing at least the fluorescent image acquisition processing (F3) during the period between the visible image acquisition processing (W2 = B2 + G2 + R2) and the next visible image acquisition processing (W4 = B4 + G4 + R4); and executing at least the ambient image acquisition processing (A5) during the period between the visible image acquisition processing (W4 = B4 + G4 + R4) and the next visible image acquisition processing (W6 = B6 + G6 + R6). The image processing unit (24) then generates a first composite image (W2 + (F3 - A1)) using at least the first visible image (W2) acquired in the first visible image acquisition processing, the fluorescent image (F3) acquired during the period after the first visible image acquisition processing, and the ambient image (A1) acquired during the period before the first visible image acquisition processing. Furthermore, the image processing unit (24) generates a second composite image (W4 + (F3 - A5)) using at least the second visible image (W4) acquired in the second visible image acquisition processing, the fluorescent image (F3) acquired during the period before the second visible image acquisition processing, and the ambient image (A5) acquired during the period after the second visible image acquisition processing. The first composite image and the second composite image are alternately output at 60 fps. During the visible image acquisition processing, the control unit (22, 26, and 27) controls the light source device (40) so that the visible light (42L) is output either only during the blanking period of the image sensor (15R, 15G, 15B) or only during a partial period around the blanking period. Furthermore, during the fluorescent image acquisition processing, the control unit (22, 26, and 27) controls the light source device (40) so that the excitation light (44L) is output throughout the entire exposure period of the image sensor (15R, 15G, and 15B).

Note that in the second embodiment, the fluorescent image and the ambient image are generated only from the output of the imaging element 15R. However, when the ambient image is corrected with respect to the visible image, or when the wavelength of fluorescence spans a plurality of imaging elements, the fluorescent image and/or the ambient image may be generated using the outputs of the plurality of imaging elements.

### Third Embodiment

Hereinafter, an imaging device according to a third embodiment will be described.

The basic configuration of the imaging device is the same as that of the second embodiment (FIG. 7). However, the three imaging elements 15R, 15G, and 15B are global-shutter-type image sensors. Furthermore, it is assumed that the imaging elements 15R, 15G, and 15B enable high-speed driving and a sensor output of 240 fps at maximum.

FIG. 11 illustrates an example of the timing of illumination, exposure, and image output according to this embodiment.

Times T0, T1, T2, etc., are arranged at 1/240-second intervals. In accordance with instructions from the timing control unit 26, the white LED 42 repeats a cycle in which it is lit at time T0, extinguished from time T1 to time T3, lit at time T4, and extinguished from time T5 to time T7 in a 4/240-second cycle. The excitation LD 44 repeats a cycle in which it is lit from time T1 to time T3, extinguished from time T4 to time T8, lit from time T9 to time Tb, and extinguished from time Tc to time Tg in a 8/240-second cycle. Periods during which both the white LED 42 and the excitation LD 44 are extinguished in the 8/240-second cycle occur, such as from time T5 to time T7 and from time Td to time Tf.

The sensors that have exposed light reflected from the white LED 42 at time T0 output, at the next time T1, images B0, G0, and R0 that form the basis of the resulting visible image W0, respectively. Next, from time T1 to time T3, exposure for three frames at 240 fps is continued as a long-exposure setting for at least the R-sensor, and the resulting fluorescent image F123 is output at the next time T4. Note that the three-digit number “123” added to the letter “F,” which represents a fluorescent image, indicates that exposure was performed during the period from time T1 to time T3. Time T2 and time T3 are blanking periods, during which no substantial output is made from the R-sensor. Similarly, exposure for three frames is continued from time T5 to time T7, and the resulting ambient image A567 is output at the next time T8. The three-digit number “567” added to the letter “A,” which represents an ambient image, indicates that exposure was performed during the period from time T5 to time T7. For the B-sensor and the G-sensor, only the images that form the basis of a visible image are acquired by discarding images during periods other than the lighting period of the white LED 42, or by restricting exposure during periods other than the lighting period of the white LED 42 by the shutter function. Here, in FIG. 11, substantially ineffective exposure timings other than the lighting period of the white LED 42 are denoted as “shutter.” As a result, the sensor outputs repeat a cycle of the visible image Wn, the fluorescent images Fn+1 to Fn+3, the visible image Wn+4, and the ambient images An+5 to An+7. The subsequent image processing flow and the resulting advantages are the same as those in the first embodiment, and their description will be omitted.

In this embodiment, the exposure times for the fluorescent image and the ambient image can be made three times that for the visible image. Therefore, this embodiment is effective when the intensity of the white light is considerably higher than the fluorescent intensity and the ambient light intensity.

The features of control of the imaging device according to this embodiment as described above can be summarized as follows. The control unit (22, 26, and 27) can execute the visible image acquisition processing to capture a visible image, the fluorescent image acquisition processing to capture a fluorescent image, and the ambient image acquisition processing to capture an ambient image by controlling the light source device (40) and the imaging unit (15R, 15G, and 15B). The image processing unit 24 generates a composite image using the visible image, the fluorescent image, and the ambient image, and outputs the composite image at a prescribed output image cycle (60 fps). The control unit (22, 26, and 27) executes the visible image acquisition processing at a cycle equal to or shorter than the output image cycle. Furthermore, the control unit (22, 26, and 27) performs control to alternately repeat: executing at least the fluorescent image acquisition processing (F123) during the period between the visible image acquisition processing (W0 = B0 + G0 + R0) and the next visible image acquisition processing (W4 = B4 + G4 + R4); and executing at least the ambient image acquisition processing (A567) during the period between the visible image acquisition processing (W4 = B4 + G4 + R4) and the next visible image acquisition processing (W8 = B8 + G8 + R8). The image processing unit (24) then generates a first composite image (W0 + (F123 - A*)) using at least the first visible image (W0) acquired in the first visible image acquisition processing, the fluorescent image (F123) acquired during the period after the first visible image acquisition processing, and an ambient image (A*) acquired during the period before the first visible image acquisition processing. Furthermore, the image processing unit (24) generates a second composite image (W4 + (F123 - A567)) using at least the second visible image (W4) acquired in the second visible image acquisition processing, the fluorescent image (F123) acquired during the period before the second visible image acquisition processing, and the ambient image (A567) acquired during the period after the second visible image acquisition processing. The first composite image and the second composite image are alternately output at 60 fps. The control unit (22, 26, and 27) performs control so that the exposure time (T1 to T3) for the fluorescent image acquisition processing is longer than the exposure time T0 for the visible image acquisition processing.

Note that in the third embodiment, an example was described in which all the three imaging elements 15B, 15G, and 15R are driven at 240 fps. However, the driving cycle of the imaging element 15R (second imaging element), which is also used for capturing both a fluorescent image and an ambient image, may be set shorter than that of the imaging elements 15G and 15B (first imaging element), which are used only for capturing a visible image. For example, only the imaging element 15R may be driven at 240 fps, while the remaining imaging elements 15G and 15B may be driven at 60 fps. In order to ensure an output image cycle (60 fps), the imaging elements 15G and 15B (first imaging elements) may be driven at a cycle equal to or shorter than the output image cycle, while the imaging element 15R (second imaging element) may be driven at a cycle shorter than twice the output image cycle. Furthermore, in this embodiment, the exposure time for the fluorescent image is set to be three times that for the visible image. However, if the exposure time for the fluorescent image is longer than that for the visible image, the advantage of enhancing the fluorescence intensity can be obtained.

### Fourth Embodiment

Hereinafter, an imaging device according to a fourth embodiment will be described.

The basic configuration of the imaging device is the same as that of the third embodiment. Similarly, the imaging elements 15B, 15G and 15R are global shutters and have an output of 240 fps.

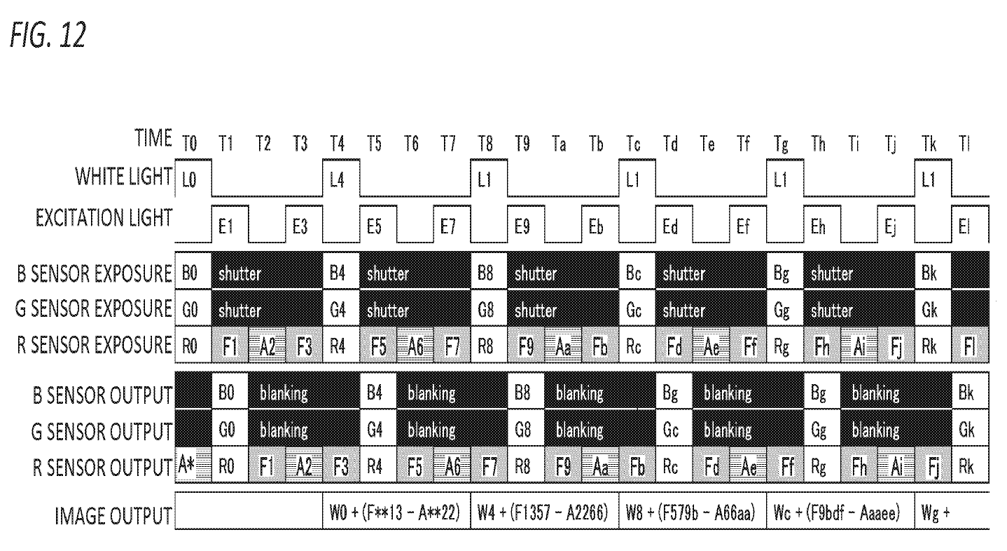

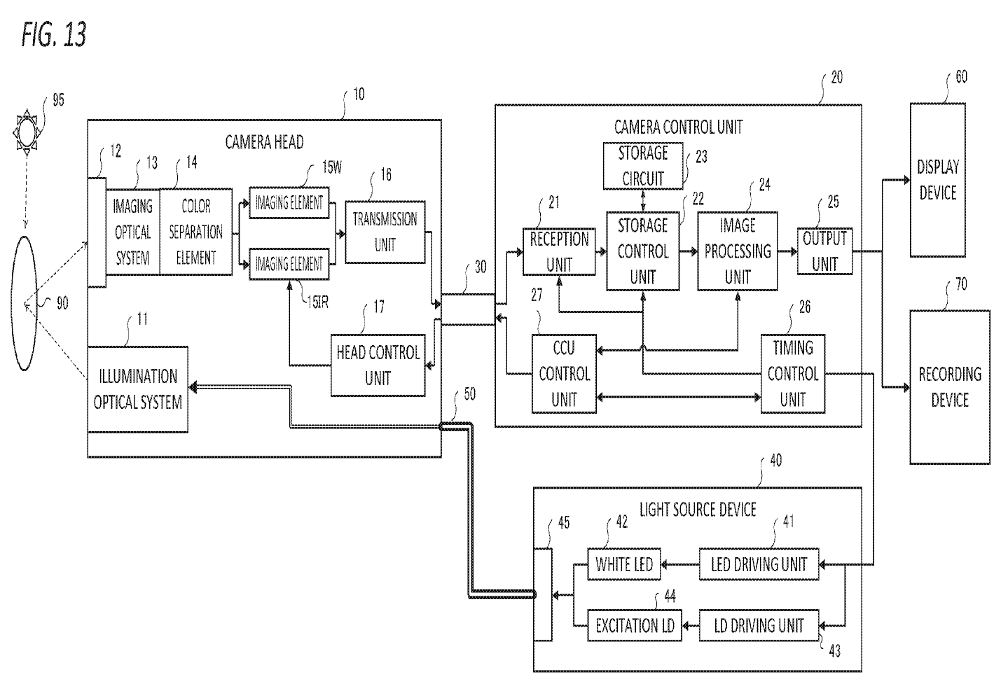

FIG. 12 illustrates an example of the timing of illumination, exposure, and image output according to this embodiment.

It is assumed that times T0, T1, T2, etc., are arranged at 1/240-second intervals. The lighting timing of the excitation LD 44 is different from that in the third embodiment (FIG. 11). The excitation LD 44 repeats a cycle in which it is lit at time T1, extinguished at time T2, lit at time T3, and extinguished at time T4 in a 2/240-second cycle. As a result, the sensor outputs differ from those of the foregoing embodiments and repeat a cycle of the visible image Wn, the fluorescent image Fn+1, the ambient image An+2, and the fluorescent image Fn+3.

The image processing unit 24 generates an output image using seven images, i.e., the visible image Wn, images of the preceding three frames (fluorescent image Fn-3, ambient image An-2, and fluorescent image Fn-1), and images of the subsequent three frames (fluorescent image Fn+1, ambient image An+2, and fluorescent image Fn+3). At this time, the time lag between the images can be as small as possible. Therefore, the ambient image of the immediately preceding or immediately subsequent frame may be subtracted from the fluorescent image. That is, the ambient image An-2 is subtracted from each of the fluorescent images Fn-3 and Fn-1, and the ambient image An+2 is subtracted from each of the fluorescent images Fn+1 and Fn+3. The subtracted images are summed to generate a pseudo-color fluorescent intensity image, which is superimposed on the visible image Wn.

For example, FIG. 12 illustrates a case in which an output image is generated using seven images, i.e., F1, A2, F3, W4 (= B4 + G4 + R4), F5, A6, and F7. In order to remove the ambient light, subtractions F1 - A2, F3 - A2, F5 - A6, and F7 - A6 are performed, and the resulting images are summed. The fluorescent intensity image representing the sum, namely, (F1 - A2) + (F3 - A2) + (F5 - A6) + (F7 - A6) is concisely denoted as (F1357 - A2266) in FIG. 12. An image in which the fluorescent intensity image is superimposed on the visible image W0, that is, W0 + (F1357 - A2266) is the final output image.

In the generation, the time lag between the visible image, the fluorescent image, and the ambient image is canceled solely by each generation result, but the spread of the marker in the motion direction does not change significantly. The advantages of this embodiment are that an output image can be generated with less time lag, and that the substantial fluorescence sensitivity is higher than that of the third embodiment. However, the requirements for the capacity and the speed of the storage circuit increase.

The features of control of the imaging device according to this embodiment as described above can be summarized as follows. The control unit (22, 26, and 27) can execute the visible image acquisition processing to capture a visible image, the fluorescent image acquisition processing to capture a fluorescent image, and the ambient image acquisition processing to capture an ambient image by controlling the light source device (40) and the imaging unit (15R, 15G, and 15B). The image processing unit (24) generates a composite image using the visible image, the fluorescent image, and the ambient image, and outputs the composite image at a prescribed output image cycle (60 fps). The control unit (22, 26, and 27) executes the visible image acquisition processing at a cycle equal to or shorter than the output image cycle. Furthermore, the control unit (22, 26,and 27) performs control to execute each of the fluorescent image acquisition processing (F5 and F7) and the ambient image acquisition processing (A6) at least once during the period between the visible image acquisition processing (W4 = B4 + G4 + R4) and the next visible image acquisition processing (W8 = B8 + G8 + R8). The image processing unit (24) then generates a composite image (W4 + (F1357 - A2266)) using at least the visible image (W4) acquired in the visible image acquisition processing, the fluorescent images (F1 and F3) and the ambient image (A2) acquired during the period before the visible image acquisition processing, and fluorescent images (F5 and F7) and the ambient image (A6) acquired during the period after the visible image acquisition processing.

Note that in the fourth embodiment as well, an example was described in which all the three imaging elements 15B, 15G, and 15R are driven at 240 fps. However, the driving cycle of the imaging element 15R (second imaging element), which is also used for capturing both a fluorescent image and an ambient image, may be set shorter than that of the imaging elements 15G and 15B (first imaging element), which are used only for capturing a visible image. For example, only the imaging element 15R may be driven at 240 fps, while the remaining imaging elements 15G and 15B may be driven at 60 fps. In order to ensure an output image cycle (60 fps), the imaging elements 15G and 15B (first imaging elements) may be driven at a cycle equal to or shorter than the output image cycle, while the imaging element 15R (second imaging element) may be driven at a cycle shorter than half of the output image cycle.

### Fifth Embodiment

FIG. 13 illustrates a configuration example of an imaging device according to a fifth embodiment.

The differences from the second embodiment (FIG. 7) are that the color separation element 14 is a two-color separation prism, and that two imaging elements 15W and 15IR are provided. The imaging element 15W is of a type that incorporates a color filter, while the imaging element 15IR is of a type that does not incorporate a color filter. This type is generally referred to as a two-chip camera. In addition, in this embodiment, it is assumed that these imaging elements are global-shutter-type image sensors.

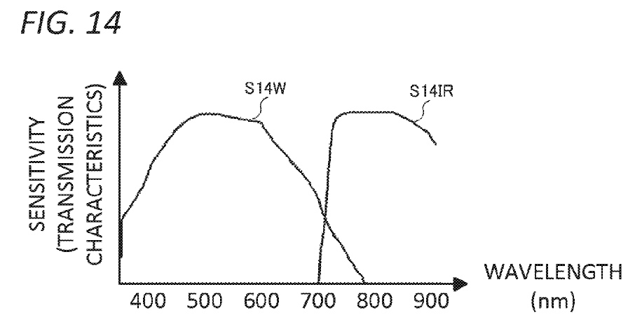

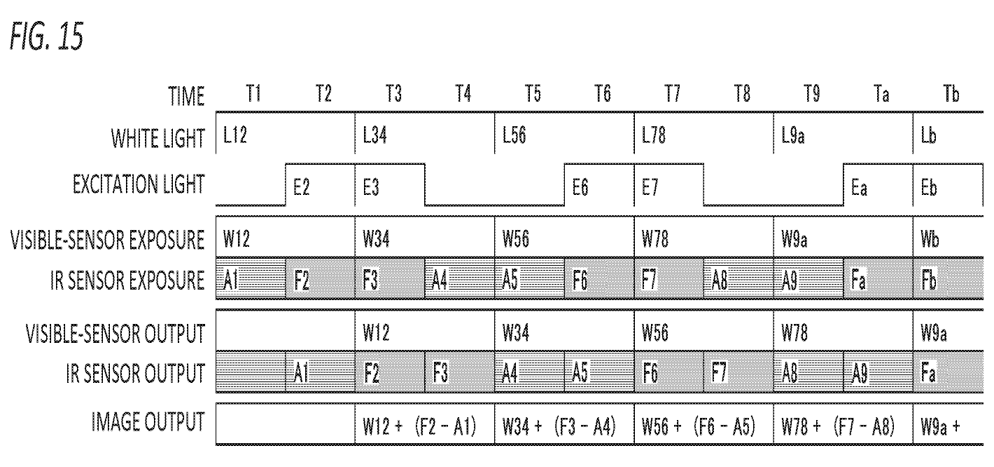

FIG. 14 illustrates an example of the spectral characteristics of the color separation element 14 according to this embodiment. The color separation element 14 separately emits light toward two emission surfaces. These two emission surfaces are designed to have the transmission characteristics indicated by S14W and S14IR, respectively. The transmission characteristics indicated by S14W mainly transmit visible light, while the transmission characteristics indicated by S14IR mainly transmit near-infrared light. The light emitted through S14W enters the imaging element 15W, and the light emitted through S14IR enters the imaging element 15IR. The characteristics of the imaging element 15W may be the same, for example, as those of the imaging element (FIG. 4) of the first embodiment, and the characteristics of the imaging element 15IR may be the same, for example, as those of the imaging element (FIG. 9) of the second embodiment. Furthermore, although the wavelength characteristics of the light source device 40 are as shown in FIG. 2, the characteristics of the ambient light 95L are considered to differ from the foregoing examples and to be widely distributed from the visible range to the near-infrared range (e.g., from 400 nm to 900 nm).

If the wavelength of the ambient light 95L falls outside the transmission range of S14IR of the color separation element 14, no ambient light mixes into a fluorescent image obtained by the imaging element 15IR. Therefore, the fluorescent image obtained by the imaging element 15IR can be used as it is as the intensity of the fluorescence 90F without any special processing. However, the ambient light 95L including its wavelength is often outside of the controllable range and may contain near-infrared light as described above. In such a case, some of the ambient light 95L passes through the emission surface of the S14IR and is received by the imaging element 15IR. When this occurs, it is no longer possible to acquire the fluorescence 90F alone.

FIG. 15 illustrates an example of the timing of illumination, exposure, and image output according to this embodiment.

It is assumed that times T1, T2, T3, etc., are arranged at 1/120-second intervals. The white LED 42 is kept constantly lit, for example. The excitation LD 44 repeats a cycle in which it is extinguished at time T1, lit at time T2, lit at time T3, and extinguished at time T4 in a 4/120-second cycle. In two frames within the 4/120-second cycle, such as times T1 and T4, the excitation LD 44 is extinguished. Therefore, there occurs a timing at which only ambient light is received by the imaging element 15IR.

The imaging element 15W is driven, for example, at 60 fps and outputs a color visible image every 1/60 second. On the other hand, the imaging element 15IR is driven at 120 fps and outputs images in a cycle of the ambient image An, the fluorescent image Fn+1, the fluorescent image Fn+2, and the ambient image An+3.

In the image processing unit 24, the ambient image An-1 or An+1 is subtracted from the fluorescent image Fn to calculate the fluorescent intensity. In addition, a cycle of the image output combined with a visible image is 60 fps. That is, in this embodiment as well, the first imaging element 15W, which is used only for capturing a visible image, is driven at the output image cycle (60 fps), and the second imaging element 15IR, which is used for capturing a fluorescent image and an ambient image, is driven at a cycle (120 fps) shorter than the output image cycle. Note that the first imaging element 15W may also be driven at a cycle shorter than the output image cycle.

As a specific example, at time T3, the ambient image A1 has already been stored in the storage circuit 23. In addition, the visible image W12 and the fluorescent image F2 have also been received from the sensor, and their storage has begun. Here, the two-digit number “12” added to the letter “W,” which represents a visible image, indicates that exposure was performed during the period from time T1 to time T2. The ambient image A1, the visible image W2, and the fluorescent image F2 are simultaneously read out at 60 fps from time T3 to time T4 and input to the image processing unit 24. The image processing unit 24 generates a fluorescent intensity image in which the component of the fluorescence 90F, which is obtained by subtracting the ambient image A1 from the fluorescent image F2, is represented in a pseudo color. The image processing unit 24 then superimposes the fluorescent intensity image on the visible image W12 to generate a desired output image, which is output at 60 fps from time T3 to time T4. Similarly, from time T5 to time T6, the fluorescent image F3, the visible image W34, and the ambient image A4 are read out at 60 fps from the storage circuit 23. The image processing unit 24 superimposes a fluorescent intensity image obtained by subtracting the ambient image A4 from the fluorescent image F3 on the visible image W34 and outputs the resulting image at 60 fps.

By such processing, the same advantage as in the first embodiment can be obtained; the time lag between the base visible image and the fluorescent image or the ambient image is canceled over the long term. However, if the ambient image and the fluorescent image are simply alternately repeated, this advantage cannot be obtained.

The features of control of the imaging device according to this embodiment as described above can be summarized as follows. The imaging unit includes the first imaging element (15W), which is used only for capturing a visible image, and the second imaging element (15IR), which is used only for capturing a fluorescent image and an ambient image. The control unit (22, 26, and 27) repeatedly executes a cycle including: the first ambient image acquisition processing (A1) for acquiring a first ambient image using the second imaging element (15IR) without outputting the excitation light (44L) from the light source device (40); the first fluorescent image acquisition processing (F2) for outputting the excitation light (44L) from the light source device (40) and acquiring a first fluorescent image using the second imaging element (15IR); the second fluorescent image acquisition processing (F3) for outputting excitation light (44L) from the light source device (40) and acquiring a second fluorescent image using the second imaging element (15IR); and second ambient image acquisition processing (A4) for acquiring a second ambient image using the second imaging element (15IR) without outputting the excitation light (44L) from the light source device (40).

Although not illustrated in the figure, a four-chip camera may be used, in which a four-color separation prism serves as the color separation element 14 and four imaging elements are employed. In this case, three imaging elements of a type that does not incorporate a color filter are used instead of the imaging element 15W.

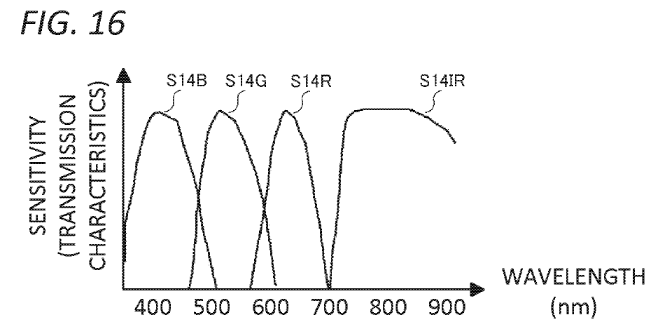

FIG. 16 illustrates an example of the spectral characteristics of the four emission surfaces of the color separation element 14 in this case. Visible light is received by the imaging elements 15B, 15G, and 15R, which are connected to the emission surfaces S14B, S14G, and S14R, respectively, and infrared light is received by the imaging element 15IR, which is connected to the emission surface S14IR.

The provision of the imaging element 15IR dedicated to infrared light, its control method, and the advantages obtained by this proposal are the same as those of the two-chip camera, and their description will be omitted.

### Modified embodiment

When the balance between the intensities of ambient light and fluorescent light differs significantly, it is effective to increase the relative intensity of the fluorescent light by reducing the transmittance of visible light. For example, the transmission characteristics of the excitation-light cut filter 12 in the visible light wavelength range may be reduced, or a separate ND filter used only for visible light may be provided. For example, an optical filter having lower transmittance in the visible light wavelength band than in the infrared light wavelength band can be provided at the front stage of the imaging optical system 13 or between the imaging optical system 13 and the imaging unit. As a result, the intensity of the fluorescence relative to that of visible light can be increased. Note that the balance of the transmittance between infrared light and visible light in the optical filter may be appropriately set in consideration of the characteristics of the light source device and the characteristics of the imaging unit.

As illustrated, the light source device has a two-color configuration with a white LED and an excitation LD, but it may alternatively have individual LEDs corresponding to R, G, B, and the excitation light may also be provided by an LED. A combination of a xenon or halogen light source and an optical filter may also be used.

Only ICG was provided as an example of fluorescent materials, but fluorescence observation using other materials such as fluorescein or 5-ALA is also covered by this proposal. The wavelength of the excitation light may be adjusted to the spectral characteristics of each fluorescent material and the imaging device. Fluorescein and 5-ALA differ from ICG in that their fluorescence is also in the visible wavelength region. However, there is still a demand for emphasizing weak fluorescence beyond what is visually perceived, and this proposal is useful in that case. In addition, when an appropriate excitation light source is selected, for example, in the configuration of the second embodiment, the excitation-light cut filter 12 can be omitted because the imaging elements for receiving the excitation light and fluorescence are separated by the color separation element.

The impact of ambient light on a visible image may be reduced on the basis of an ambient image. For this purpose, as in the case of a fluorescent image, a method such as subtracting an ambient image having a less time lag may be employed. This is particularly effective in cases where automatic brightness control of a visible image is performed by adjusting the intensity of white light, and where automatic brightness control of a fluorescent intensity image is performed using the ratio of the white light to a reflected component. In this way, by subtracting an ambient image from a visible image to reduce the impact of ambient light on the visible image, accurate automatic brightness control can be performed with suppressed calculation error.

Furthermore, the present disclosure is not limited to the above-described embodiments as such, and can be embodied by modifying components within a scope not departing from the gist of the present disclosure at the implementation stage.

Note that although the above description mainly concerns the imaging device, the present disclosure is also applicable to an imaging method that performs the same processing as the imaging device, an image processing program that causes a computer to perform the same processing as the imaging device, a non-transitory computer-readable recording medium that records the image processing program, and the like.

Note that the above-described various types of control may be processing that is carried out by one piece of hardware (e.g., processor or circuit), or otherwise. Processing may be shared among a plurality of pieces of hardware (e.g., a plurality of processors, a plurality of circuits, or a combination of one or more processors and one or more circuits), thereby carrying out the control of the entire device.

Also, the above processor is a processor in the broad sense, and includes general-purpose processors and dedicated processors. Examples of general-purpose processors include a central processing unit (CPU), a micro processing unit (MPU), a digital signal processor (DSP), and so forth. Examples of dedicated processors include a graphics processing unit (GPU), an application-specific integrated circuit (ASIC), a programmable logic device (PLD), and so forth. Examples of PLDs include a field-programmable gate array (FPGA), a complex programmable logic device (CPLD), and so forth.

The embodiment described above (including variation examples) is merely an example. Any configurations obtained by suitably modifying or changing some configurations of the embodiment within the scope of the subject matter of the present disclosure are also included in the present disclosure. The present disclosure also includes other configurations obtained by suitably combining various features of the embodiment.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a 'non-transitory computer-readable storage medium') to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)^{TM}), a flash memory device, a memory card, and the like.

According to the present disclosure, it is possible to suppress a decrease in the frame rate of visible light images while enabling fluorescence observation under ambient light, thereby providing moving images suitable for users.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-024787, filed February 19, 2025, which is hereby incorporated by reference herein in its entirety.

What is claimed is:

1. An imaging device comprising:
a light source module capable of outputting visible light and excitation light;
an imaging module having at least one image sensor;
a processor; and
a memory storing a program which, when executed by the processor, causes the imaging device to
control the light source module and the imaging module, thereby enabling visible image acquisition processing to capture a visible image representing an image of a subject using visible light output from the light source module, fluorescent image acquisition processing to capture a fluorescent image representing an image of fluorescence generated in the subject using excitation light output from the light source module, and ambient image acquisition processing to capture an ambient image representing an image of the subject using ambient light, and
execute image processing to (i) generate a composite image using the visible image, the fluorescent image, and the ambient image, and (ii) output the composite image at a prescribed output image cycle, wherein
the visible image acquisition processing is executed at a cycle equal to or shorter than the output image cycle, wherein
executing at least the fluorescent image acquisition processing during a period between the visible image acquisition processing and the next visible image acquisition processing and executing at least the ambient image acquisition processing during a period between the visible image acquisition processing and the next visible image acquisition processing are alternately repeated, and wherein
the image processing comprises:,
processing to generate a first composite image using at least a first visible image acquired in first visible image acquisition processing, a fluorescent image acquired during a period after the first visible image acquisition processing, and an ambient image acquired during a period before the first visible image acquisition processing, and
processing to generate a second composite image using at least a second visible image acquired in second visible image acquisition processing, a fluorescent image acquired during a period before the second visible image acquisition processing, and an ambient image acquired during a period after the second visible image acquisition processing.

2. The imaging device according to claim 1, wherein in the image processing, the first composite image and the second composite image are alternately output.

3. The imaging device according to claim 1, wherein the output image cycle is at least 50 fps.

4. The imaging device according to claim 1, wherein the imaging module includes an image sensor having sensitivity to a wavelength of the fluorescence, and
a cycle including following processing 1) to 4) is repeatedly executed:
1) first visible image acquisition processing in which only visible light is output from the light source module and a first visible image is acquired by the imaging module;
2) fluorescent image acquisition processing in which only excitation light is output from the light source module and a fluorescent image is acquired by the imaging module;
3) second visible image acquisition processing in which only visible light is output from the light source module and a second visible image is acquired by the imaging module; and
4) ambient image acquisition processing in which an ambient image is acquired by the imaging module without outputting light from the light source module.

5. The imaging device according to claim 1, wherein the image sensor is a rolling-shutter-type image sensor, and
the program, when executed by the processor, further causes the imaging device to
execute control processing to control the light source module so that, in the visible image acquisition processing, the visible light is output during a blanking period of the image sensor or during a partial period around the blanking period.

6. The imaging device according to claim 5 wherein in the control processing, the light source module is controlled so that, in the fluorescent image acquisition processing, the excitation light is output over an entire exposure period of the image sensor.

7. The imaging device according to claim 1, wherein the program, when executed by the processor, further causes the imaging device to execute control processing so that an exposure time of the fluorescent image acquisition processing is longer than an exposure time of the visible image acquisition processing.

8. The imaging device according to claim 1, wherein in the image processing, the ambient image is subtracted from the fluorescent image in the processing to generate the composite image, thereby reducing an ambient light component contained in the fluorescent image.

9. The imaging device according to claim 8 wherein in the image processing, the fluorescent image, in which the ambient light component has been reduced, is superimposed on the visible image using a pseudo color in the processing to generate the composite image.

10. The imaging device according to claim 1, wherein in the image processing, the ambient image is subtracted from the visible image in the processing to generate the composite image, thereby reducing an ambient light component contained in the visible image.

11. The imaging device according to claim 1, further comprising an optical filter having lower transmittance in a visible light wavelength band than in an infrared light wavelength band.

12. An imaging device comprising:
a light source module capable of outputting visible light and excitation light;
an imaging module having at least one image sensor;
a processor; and
a memory storing a program which, when executed by the processor, causes the imaging device to
control the light source module and the imaging module, thereby enabling visible image acquisition processing to capture a visible image representing an image of a subject using visible light output from the light source module, fluorescent image acquisition processing to capture a fluorescent image representing an image of fluorescence generated in the subject using excitation light output from the light source module, and ambient image acquisition processing to capture an ambient image representing an image of the subject using ambient light, and
execute image processing to (i) generate a composite image using the visible image, the fluorescent image, and the ambient image and (ii) output the composite image at a prescribed output image cycle, wherein
the imaging module has a first image sensor used only for capturing the visible image and a second image sensor used for capturing at least the fluorescent image and the ambient image,
the first image sensor is driven so that the visible image is acquired at a cycle equal to or shorter than the output image cycle, and
the second image sensor is driven at a cycle shorter than the output image cycle.

13. The imaging device according to claim 12 wherein the second image sensor is driven at a cycle shorter than half of the output image cycle.

14. The imaging device according to claim 12 wherein the second image sensor is also used for capturing the visible image, and
the program, when executed by the processor, further causes the imaging device to execute control processing so that an exposure time of the second image sensor in the fluorescent image acquisition processing is longer than an exposure time of the second image sensor in the visible image acquisition processing.

15. The imaging device according to claim 12 wherein the second image sensor is used only for capturing the fluorescent image and the ambient image, and
a cycle including following processing 1) to 4) is repeatedly executed:
1) first ambient image acquisition processing in which a first ambient image is acquired by the second image sensor without outputting excitation light from the light source module;
2) first fluorescent image acquisition processing in which excitation light is output from the light source module and a first fluorescent image is acquired by the second image sensor;
3) second fluorescent image acquisition processing in which excitation light is output from the light source module and a second fluorescent image is acquired by the second image sensor; and
4) second ambient image acquisition processing in which a second ambient image is acquired by the second image sensor without outputting excitation light from the light source module.

16. An imaging device comprising:
a light source module capable of outputting visible light and excitation light;
an imaging module having at least one image sensor;
a processor; and
a memory storing a program which, when executed by the processor, causes the imaging device to
control the light source module and the imaging module, thereby enabling visible image acquisition processing to capture a visible image representing an image of a subject using visible light output from the light source module, fluorescent image acquisition processing to capture a fluorescent image representing an image of fluorescence generated in the subject using excitation light output from the light source module, and ambient image acquisition processing to capture an ambient image representing an image of the subject using ambient light, and
execute image processing to (i) generate a composite image using the visible image, the fluorescent image, and the ambient image and (ii) output the composite image at a prescribed output image cycle, wherein
the visible image acquisition processing is executed at a cycle equal to or shorter than the output image cycle, wherein
each of the fluorescent image acquisition processing and the ambient image acquisition processing is executed at least once during a period between the visible image acquisition processing and the next visible image acquisition processing, and wherein
in the image processing, a composite image is generated using at least a visible image acquired in the visible image acquisition processing, a fluorescent image and an ambient image acquired during a period before the visible image acquisition processing, and a fluorescent image and an ambient image acquired during a period after the visible image acquisition processing.

17. A control method of an imaging device including a light source module capable of outputting visible light and excitation light, and an imaging module having at least one image sensor,
the control method comprising:
executing visible image acquisition processing to capture a visible image representing an image of a subject using visible light output from the light source module;
executing fluorescent image acquisition processing to capture a fluorescent image representing an image of fluorescence generated in the subject using excitation light output from the light source module;
executing ambient image acquisition processing to capture an ambient image representing an image of the subject using ambient light;
generating a composite image using the visible image, the fluorescent image, and the ambient image; and
outputting the composite image at a prescribed output image cycle, wherein
the visible image acquisition processing is executed at a cycle equal to or shorter than the output image cycle, wherein
executing at least the fluorescent image acquisition processing during a period between the visible image acquisition processing and the next visible image acquisition processing and executing at least the ambient image acquisition processing during a period between the visible image acquisition processing and the next visible image acquisition processing are alternately repeated, and wherein
generating the composite image includes
processing to generate a first composite image using at least a first visible image acquired in first visible image acquisition processing, a fluorescent image acquired during a period after the first visible image acquisition processing, and an ambient image acquired during a period before the first visible image acquisition processing, and
processing to generate a second composite image using at least a second visible image acquired in second visible image acquisition processing, a fluorescent image acquired during a period before the second visible image acquisition processing, and an ambient image acquired during a period after the second visible image acquisition processing.

18. A control method of an imaging device including a light source module capable of outputting visible light and excitation light, and an imaging module having at least one image sensor,
the control method comprising:
executing visible image acquisition processing to capture a visible image representing an image of a subject using visible light output from the light source module;
executing fluorescent image acquisition processing to capture a fluorescent image representing an image of fluorescence generated in the subject using excitation light output from the light source module;
executing ambient image acquisition processing to capture an ambient image representing an image of the subject using ambient light;
generating a composite image using the visible image, the fluorescent image, and the ambient image; and
outputting the composite image at a prescribed output image cycle, wherein
the imaging module has a first image sensor used only for capturing the visible image and a second image sensor used for capturing at least the fluorescent image and the ambient image,
the first image sensor is driven so that the visible image is acquired at a cycle equal to or shorter than the output image cycle, and
the second image sensor is driven at a cycle shorter than the output image cycle.

19. A control method of an imaging device including a light source module capable of outputting visible light and excitation light, an imaging module having at least one image sensor,
the control method comprising:
executing visible image acquisition processing to capture a visible image representing an image of a subject using visible light output from the light source module;
executing fluorescent image acquisition processing to capture a fluorescent image representing an image of fluorescence generated in the subject using excitation light output from the light source module;
executing ambient image acquisition processing to capture an ambient image representing an image of the subject using ambient light;
generating a composite image using the visible image, the fluorescent image, and the ambient image; and
outputting the composite image at a prescribed output image cycle, wherein
the visible image acquisition processing is executed at a cycle equal to or shorter than the output image cycle, wherein
each of the fluorescent image acquisition processing and the ambient image acquisition processing is executed at least once during a period between the visible image acquisition processing and the next visible image acquisition processing, and wherein
the composite image is generated using at least a visible image acquired in the visible image acquisition processing, a fluorescent image and an ambient image acquired during a period before the visible image acquisition processing, and a fluorescent image and an ambient image acquired during a period after the visible image acquisition processing.
